(19) United States
(12) Patent Application Publication
(10) Pub. No.: US 2019/0112346 A1
(43) Pub. Date: Apr. 18, 2019

(54) COMPOSITION HAVING COVERED FIBROUS PROTEIN ANTIGENIC PROPERTIES AND PRODUCTION METHOD THEREFOR

(71) Applicants: JAPAN FISHERIES RESEARCH AND EDUCATION AGENCY, Yokohama-shi, Kanagawa (JP); WAKO FILTER TECHNOLOGY Co., LTD., Tokyo (JP)

(72) Inventors: Masakatsu Usui, Shimonoseki-shi, Yamaguchi (JP); Taiko Miyasaki, Kitakyushu-shi, Fukuoka (JP); Akira Saito, Toride-shi, Ibaraki (JP)

(21) Appl. No.: 16/089,253

(22) PCT Filed: Mar. 28, 2017
(86) PCT No.: PCT/JP2017/012623

§ 371 Date: Sep. 27, 2018

(30) Foreign Application Priority Data
Mar. 28, 2016 (JP) 2016-063157

(51) Int. Cl. C07K 14/47

(52) U.S. Cl. CPC C07K 14/4716; C07K 1/00

(57) ABSTRACT

The present invention is a novel composition including a fibrous protein having covered antigenicity, and the composition contains a complex prepared by bonding a fibrous protein such as tropomyosin derived from shrimp to a maltooligosaccharide such as maltopentaose or maltoheptaose through a Maillard reaction.

### TECHNICAL FIELD

The present invention relates to a novel composition including a fibrous protein having covered antigenicity and a method for producing the composition.

### BACKGROUND ART

A method of masking the antigenic structure of a globular protein by Maillard reaction of a globular protein such as cedar pollen allergen and a polysaccharide such as galactomannan has been disclosed (for example, Patent Document 1).

### CITATION LIST

### Patent Literature

Patent Document 1: JP-A No. 2006-340658

### SUMMARY OF INVENTION

### Technical Problem

However, the effectiveness of the above method has not been made clear for fibrous proteins having no complicated higher order structure, such as tropomyosin derived from shrimp. The inventors of the present application have carried out a Maillard reaction of galactomannan and tropomyosin but have failed to observe reduction of the antigenic structure.

Meanwhile, the inventors of the present application have carried out various studies to reduce the antigenicity of fibrous proteins and have found saccharides that can reduce the antigenicity of a fibrous protein.

On the basis of the above finding by the inventors of the present application, the present invention is intended to provide a novel composition including a fibrous protein having covered antigenicity.

### Solution to Problem

In other words, a composition including a fibrous protein having covered antigenicity pertaining to the present invention includes a complex prepared by bonding a fibrous protein to a maltooligosaccharide.

In the composition, the fibrous protein is preferably tropomyosin derived from shrimp, for example.

In the composition, the maltooligosaccharide preferably has a molecular size between maltopentaose (a molecular weight of about 829) as a pentasaccharide (an oligosaccharide composed of five monosaccharide molecules) and maltoheptaose (a molecular weight of about 1,153) as a heptasaccharide.

A method for producing a composition including a fibrous protein having covered antigenicity pertaining to the present invention includes subjecting a fibrous protein and a maltooligosaccharide to a Maillard reaction.

In the method, the Maillard reaction is preferably performed by dry-heating a mixture of the fibrous protein and the maltooligosaccharide for 6 hours or more.

### Advantageous Effects of Invention

According to the present invention, the antigenicity of a fibrous protein can be reduced by bonding a fibrous protein to a maltooligosaccharide through Maillard reaction.

### BRIEF DESCRIPTION OF DRAWINGS

FIG. 1 show results of SDS-PAGE analysis of Pen j1 after dry-heating (panel A), results of dot blot analysis (panel B), and results of binding degree to IgE antibody (panel C).

FIG. 2 shows results of SDS-PAGE analysis of a complex of Pen j1 and glucose (panel A), results of dot blot analysis (panel B), and results of binding degree to IgE antibody (panel C).

FIG. 3 show results of SDS-PAGE analysis of a complex of Pen j1 and D-(+)-maltotriose (panel A), results of dot blot analysis (panel B), and results of binding degree to IgE antibody (panel C).

FIG. 4 show results of SDS-PAGE analysis of a complex of Pen j1 and maltopentaose (panel A), results of dot blot analysis (panel B), and results of binding degree to IgE antibody (panel C).

FIG. 5 show results of SDS-PAGE analysis of a complex of Pen j1 and maltoheptaose (panel A), results of dot blot analysis (panel B), and results of binding degree to IgE antibody (panel C).

FIG. 6 show results of SDS-PAGE analysis of a complex of Pen j1 and galactomannan (panel A), results of dot blot analysis (panel B), and results of binding degree to IgE antibody (panel C).

### DESCRIPTION OF EMBODIMENTS

The present invention will now be described in detail.

A composition including a fibrous protein having covered antigenicity pertaining to the present embodiment contains a complex prepared by bonding a fibrous protein to a maltooligosaccharide.

As the fibrous protein, tropomyosin derived from shrimp can be used. The tropomyosin derived from shrimp has a molecular weight of about 35 kDa and is a fibrous protein mainly having an α-helix structure. The tropomyosin is highly homologous among crustaceans, mollusks, insects, and the like and can be highly crossed.

Other fibrous proteins can include collagen, myosin, keratin, and elastin.

As the maltooligosaccharide, maltopentaose or maltoheptaose can be used.

The composition including a fibrous protein having covered antigenicity in the present embodiment can be produced through a Maillard reaction by dry-heating a mixture of tropomyosin as the fibrous protein and maltopentaose or maltoheptaose as the maltooligosaccharide for 6 hours or more.

A specific production method will be described below.

Tropomyosin and maltopentaose (maltoheptaose) at a molar ratio of tropomyosin:maltopentaose (maltoheptaose) of about 1:50 (shrimp tropomyosin:sugar) are mixed and dissolved in water to give an aqueous solution having a tropomyosin total content of 0.1% by mass. The obtained aqueous solution is freeze-dried into a powder. The obtained powder is subjected to a Maillard reaction at a temperature of 50 to 80° C., more preferably 55 to 65° C., and a relative humidity of 30 to 50%, more preferably 30 to 40%, for 6 hours or more, and the complex of the present invention can be produced.

In the present embodiment, how many sugar molecules can be bonded to a protein molecule is determined (estimated from an amino acid sequence) to estimate the reaction efficiency from the structures and molecular weights of both molecules, and the molar ratio is set. Shrimp tropomyosin and a maltooligosaccharide have substantially uniform molecular weights, and this enables the above setting. In the embodiment, shrimp tropomyosin has 25 lysine residues exposed on the molecular surface (amino acid residues capable of being bonded to a sugar) and has high reaction efficiency with an oligosaccharide. In consideration of this, 50 molecules of an oligosaccharide, which is twice the number of lysine residues (25 residues) of a shrimp tropomyosin molecule, are to be added. In other words, tropomyosin:oligosaccharide is 1 mol:50 mol. A different substance (in molecular weight) has a different mass per mole, and thus when a solution is prepared in terms of molar ratio or molarity, the concentration (% by mass) of the solution is not constant. In consideration of solution viscosity or the like affecting the solubility or the uniformity of a protein, the total protein content in an aqueous solution is preferably about 0.1%.

### Examples

Experimental results of an epitope covering effect by sugar modification of tropomyosin derived from shrimp will be described.

Tropomyosin derived from shrimp was extracted from the edible part of penaeid shrimp under unheated conditions and was purified by isoelectric precipitation, ammonium sulfate fractionation, and ion-exchange chromatography.

For the Maillard-type sugar modification of the resulting tropomyosin (Pen j1), glucose was used as a monosaccharide, D-(+)-maltotriose, maltopentaose, and maltoheptaose were used as maltooligosaccharides, and galactomannan was used as a polysaccharide.

Low molecular weight sugars (except galactomannan) were mixed at Pen j1:sugar=1:50, whereas galactomannan was mixed at 10 times the mass of Pen j1, and each mixture was dry-heated in a thermo-hygrostat at 60° C. at a relative humidity of 35%. The sugar modification was observed by sodium dodecyl sulfate polyacrylamide gel electrophoresis (SDS-PAGE) analysis, and the epitope covering effect was evaluated by dot blot analysis using patient serum to determine the binding degree to IgE antibody.

The SDS-PAGE analysis was performed in accordance with a common Laemmli method. To a 0.1% protein solution, the same amount of a sample treatment liquid was added to give a sample for electrophoresis. As the electrophoresis gel, a precast gel, e-PAGEL (a gel concentration of 12.5%, ATTO CORPORATION, Tokyo) was used. The electrophoresis was performed using EzRun, a buffer solution for electrophoresis (ATTO CORPORATION) at 20 mA. After electrophoresis, protein was identified by Coomassie staining with Ez Stain Aqua (ATTO CORPORATION).

The antigenicity of a complex was evaluated by dot blot analysis. A PVDF membrane (Hybond-p, GE Healthcare Japan, Tokyo) was immersed in methanol for hydrophilization, then was immersed in PBS once, and was washed. To the dried PVDF membrane, 10 μl of each sample was spotted. The PVDF membrane was dried, then was immersed in a PBS-Tween 20 containing a 5% ECL Prime Blocking Reagent, and was shaken at room temperature for 1 hour for blocking. The blocked PVDF membrane was rinsed with PBS and then was washed with PBS-Tween 20. On the washed PVDF membrane, 2 ml of a shrimp allergic patient serum (22515-JH) diluted 50-fold with PBS-Tween 20 was added and reacted at room temperature for 1 hour. After the reaction, the membrane was washed with PBS-Tween 20, then 2 ml of an anti-Human IgE Antibody-HRP diluted 4,000-fold with PBS-Tween 20 was added and reacted for 1 hour, and the membrane was washed with PBS-Tween 20. Onto the PVDF membrane, a luminescence substrate (ECL Prime Western Blotting Detection Reagent, GE Healthcare Japan) was added and reacted for 5 minutes. Chemiluminescence by the antigen-antibody reaction was recorded with ChemiDoc XRS+(Bio-Rad Laboratories, Inc., CA, USA). The dot blot analysis was performed in a similar manner where the shrimp allergic patient serum was changed (22429-HL, 22596-AW).

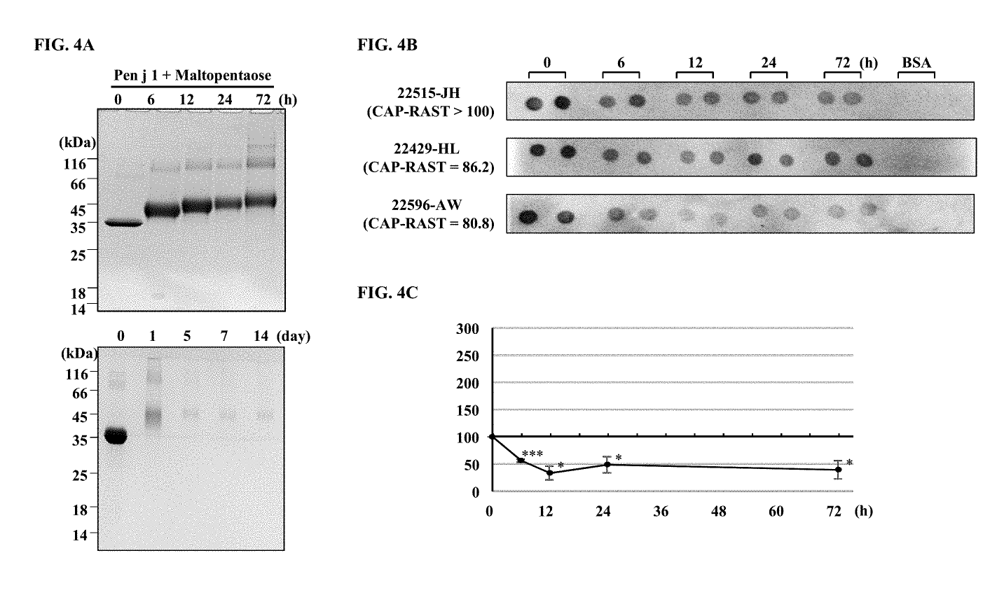

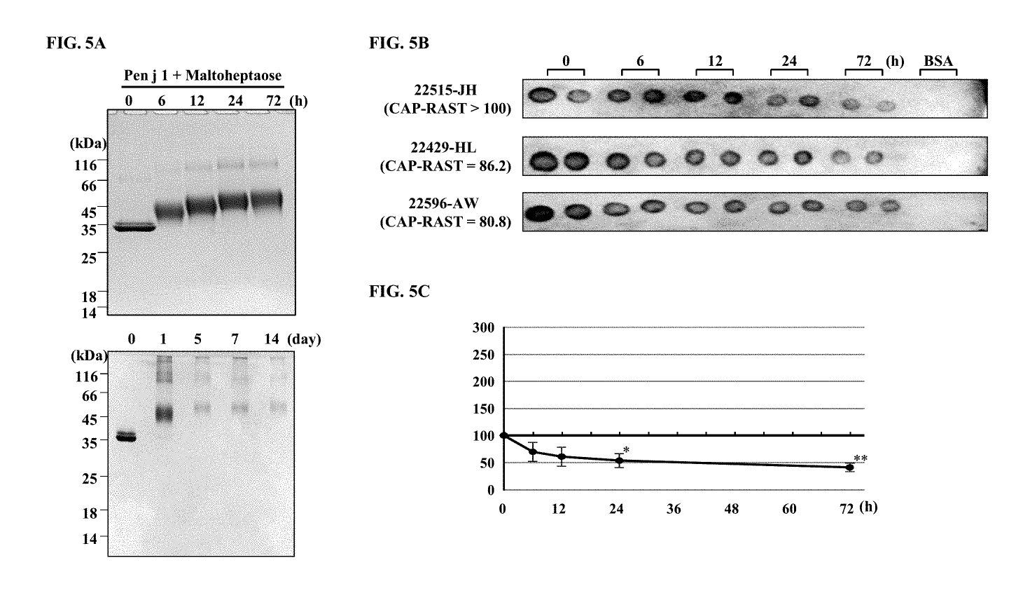

The binding degree to the IgE antibody was determined by digitalization of the density of each spot through densitometric analysis of the dot blot analysis image recorded with ChemiDoc XRS+. The average of eight positions in each spot was calculated, and the average of the corresponding complex was calculated to give the binding degree of the complex. The binding degree of each mixture at 0 hour after dry-heating was regarded as 100%, and the binding degrees of the respective dry-heating times were plotted on a graph and were subjected to statistical analysis by t test to determine significant differences in increase or decrease. In FIG. 4 and FIG. 5, “***” represents a p value of less than 0.005, “*” represents a p value of less than 0.01, and “*” represents a p value of less than 0.05.

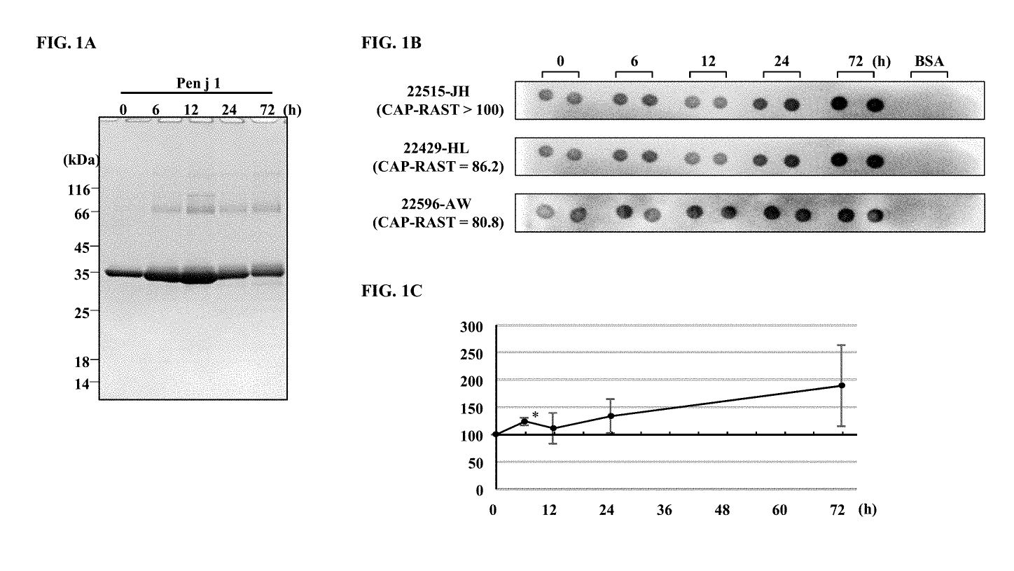

FIG. 1 show the results of SDS-PAGE analysis of Pen j1 after dry-heating (panel A), the results of dot blot analysis (panel B), and the results of binding degree to IgE antibody (panel C).

FIG. 2 shows the results of SDS-PAGE analysis of a complex of Pen j1 and glucose (panel A).

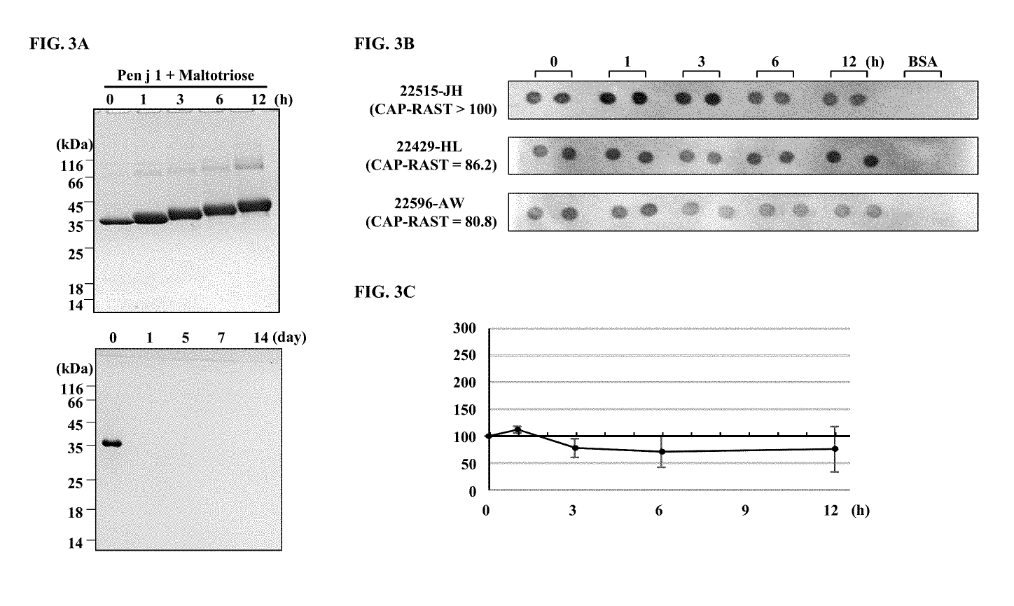

FIG. 3 show the results of SDS-PAGE analysis of a complex of Pen j1 and D-(+)-maltotriose (panel A), the results of dot blot analysis (panel B), and the results of binding degree to IgE antibody (panel C).

FIG. 4 show the results of SDS-PAGE analysis of a complex of Pen j1 and maltopentaose (panel A), the results of dot blot analysis (panel B), and the results of binding degree to IgE antibody (panel C).

FIG. 5 show the results of SDS-PAGE analysis of a complex of Pen j1 and maltoheptaose (panel A), the results of dot blot analysis (panel B), and the results of binding degree to IgE antibody (panel C).

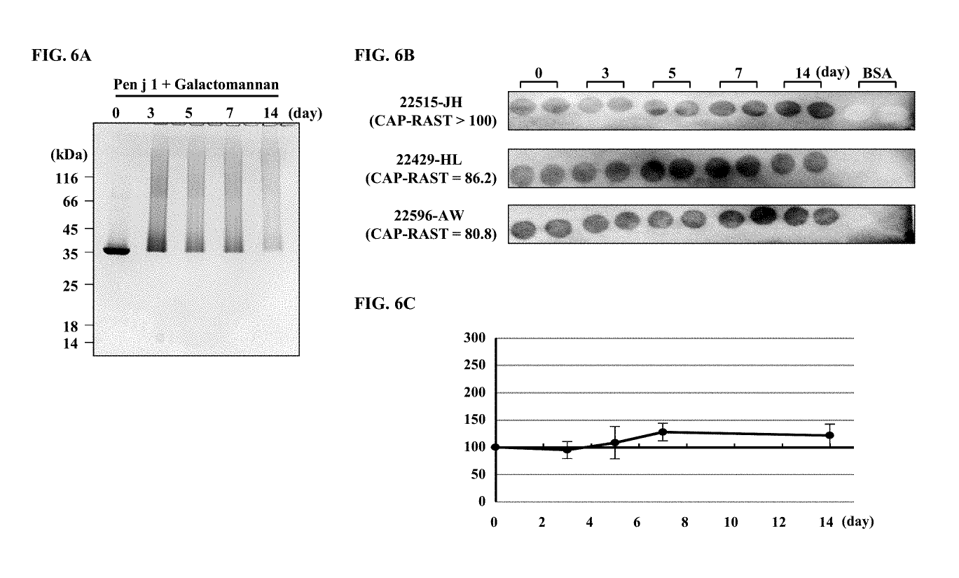

FIG. 6 show the results of SDS-PAGE analysis of a complex of Pen j1 and galactomannan (panel A) and the results of dot blot analysis (panel B).

As shown in FIG. 2, in the complex of Pen j1 and glucose, an increase in the molecular weight of Pen j1 by several kilodaltons was observed at 3 hours after dry-heating, but the samples after that were failed to be analyzed due to marked insolubilization. Similarly, as shown in FIG. 3, in the complex of Pen j1 and D-(+)-maltotriose, all the dry-heated samples underwent insolubilization.

As shown in FIG. 4 and FIG. 5, the complex of Pen j1 and maltopentaose and the complex of Pen j1 and maltoheptaose showed polymerization of Pen j1 from day 1 to day 14. As shown in FIG. 6, the complex of Pen j1 and galactomannan showed polymerization of Pen j1 in a large range from about 35 kDa to about 200 kDa or more from day 3 to day 14.

As shown in FIG. 4 and FIG. 5, the complex of Pen j1 and maltopentaose and the complex of Pen j1 and maltoheptaose showed significant decreases in the binding rate to anti-Pen j1 IgE antibody from 6 hours after dry-heating, and the decreases reached about 40% relative to the unmodified shrimp tropomyosin (100%).

### INDUSTRIAL APPLICABILITY

According to the present invention, the antigenicity of a fibrous protein can be reduced by bonding a fibrous protein to a maltooligosaccharide through a Maillard reaction.

1. A composition including a fibrous protein having covered antigenicity, the composition comprising:
a complex comprising a fibrous protein bonded to a maltooligosaccharide.

2. The composition including a fibrous protein having covered antigenicity according to claim 1, wherein the fibrous protein is tropomyosin.

3. The composition including a fibrous protein having covered antigenicity according to claim 2, wherein the tropomyosin is derived from shrimp.

4. The composition including a fibrous protein having covered antigenicity according to claim 1, wherein the maltooligosaccharide has a molecular size between maltopentaose as a pentasaccharide and maltoheptaose as a heptasaccharide.

5. A method for producing a composition including a fibrous protein having covered antigenicity, the method comprising:
subjecting a fibrous protein and a maltooligosaccharide to a Maillard reaction.

6. The method for producing a composition including a fibrous protein having covered antigenicity according to claim 5, wherein the Maillard reaction is performed by dry-heating a mixture of the fibrous protein and the maltooligosaccharide for 6 hours or more.
